(19) United States
(12) Patent Application Publication
(10) Pub. No.: US 2009/0209964 A1
(43) Pub. Date: Aug. 20, 2009

(54) Trephine designed for removal of a bone core and equipped with a device guiding it inside the bone and combining a drill bit and tubular cutting tool

(76) Inventor: Jean-Claude Yeung, Joinville Le Pont (FR)

Correspondence Address: HEDMAN & COSTIGAN P.C., 1185 AVENUE OF THE AMERICAS, NEW YORK, NY, 10036, US

(21) Appl. No.: 12/308,389

(22) PCT Filed: Jun. 12, 2007
(86) PCT No.: PCT/FR2007/000971

§ 371 Date: Feb. 9, 2009

(30) Foreign Application Priority Data
Jun. 14, 2006 (FR) 06/05291
Nov. 22, 2006 (FR) 06/10213

(51) Int. Cl. A61F 5/00

(52) U.S. Cl. 606/87

(57) ABSTRACT

A trephine for removal of a bone core comprising a tubular tool and a gripping shaft allowing the tool to be driven in rotation by an instrument, means for guiding the tool comprising a rod which protrudes from the tool and which is provided with means for centering on the trephine, the guiding means comprising a drill bit for forming a preliminary hole in the bone substance before the tool comes in contact with the bone core which hole forms a channel receiving the protruding end of the rod which constitutes a guide finger as a peg capable of being introduced inside the preliminary hole.

### TECHNICAL FIELD OF THE INVENTION

The invention is in the field of surgical instruments, and more particularly in the field of trephines for the removal of a cohesive core of bone tissue. The invention relates to such a trephine, equipped with means to facilitate its use and particularly to facilitate the tasks of the medical practitioner related to the guiding of a tubular cutting tool that comprises the trephine and the withdrawal of the bone core removed by means of such trephine.

### PRIOR ART

Known in the field of surgery are trephines for the removal of bone tissue, which are used for example in dental surgery. A first type of trephine is designed for the removal of bone tissue in the form of powdered and/or ground material. Such a trephine allows the easy use of the removed bone tissue, but the consistency of this bone tissue is unsuitable for use in bone grafts and/or filling. A second type of trephine is designed for the removal of a bone core, all or part of which can be used subsequently by the medical practitioner for bone grafts and/or filling due to its cohesive nature. The invention concerns a trephine of this second type.

The trephine is primarily composed of a tubular shaped tool the distal end of which is provided with teeth for the perforation of the bone tissue along a circular cutting line. The axial bore of the tool forms a reserve in which the core is accommodated after removal. The proximal end of the tool is provided with a shaft to be gripped by the chuck of an instrument to drive the tool in rotation. Overall, this gripping shaft is composed of an axial stem that is integral with the tool and grippable by the instrument.

The tool usually includes at its periphery at least one lateral aperture for access to the removed bone, in order to provide the medical practitioner with a passage through which the core can be withdrawn by pushing it out of the trephine after removal. After releasing the tool from its grip by the instrument that drives it in rotation, the extraction of the core from the tool is usually performed by the medical practitioner by means of a probe, a cannula or similar device, which is inserted through the aperture to exert a thrust upon the core and cause its ejection through the distal opening of the tool. Such an ejection operation is long and difficult to perform because of the fragility of the core and the risk of its damage by the cannula passing through the aperture of the tool. The core is often blocked by friction inside the trephine, especially if the core removal has been done under conditions of high temperatures.

In practice, the skill of the medical practitioner performing the perforation of the bone tissue by the trephine is decisive. More particularly, the tool has a considerable diameter for receiving in its internal bore the core to be removed, and its teeth are made on the edge of its distal end and are susceptible to being irregular. The point of impact and the path followed by the tool in the bone tissue are predetermined by the medical practitioner, and the path actually traveled by the tool depends on the skill of said practitioner in handling the trephine. This skill is particularly put to the test because of the considerable diameter of the tool, and risks of skidding and/or slipping of the tool when it is placed in contact with the bone tissue. The result is difficulty for the medical practitioner to maintain the point of impact he has selected on the cortical bone, and to maintain the predetermined path of the tool because of its considerable size.

In order to facilitate the medical practitioner's task, it has been proposed to provide the trephine with a device for guiding a tubular tool when it is placed in contact with the bone tissue. For example, according to U.S. Pat. No. 5,632,747 (SCARBOROUGH), it is proposed to provide the trephine with a guide device comprising a rod for pricking the bone tissue in such a way that the medical practitioner can ensure that the trephine has a purchase on the bone prior to placing the tool in contact against the patient's tissues. More particularly, this rod is provided at its distal end with a point that constitutes a pivot and anchoring point for the trephine prior to the insertion of the tool into the bone tissue. This rod is slidably mounted on the trephine in such a way that it can retract in accordance with the progress of the tool in the bone tissue. The tool is mounted by screwing on the guide device, which is provided with the shaft to be gripped by the chuck of the instrument to drive the tool in rotation. Moreover, the rod is used to extract the bone core from the tool. Upon completion of the removal, because the rod protrudes from the distal end of the tool, pressure exerted by the medical practitioner on the rod allows the removed core to be expelled from the tool.

Such a guide device remains unsatisfactory and requires improvement. Indeed, the purchase of the trephine on the bone does not ensure that it is sufficiently held prior to the placement of the tool in contact against the bone, because said contact generates a reaction torque on the tool that can cause the point to skid. Moreover, the aiming obtained by means of the rod does not offer any solution with respect to the axial guiding of the tool during the removal of the bone core, there being a risk of tilting the tool away from the path initially planned by the medical practitioner. Furthermore, the methods provided for extracting the removed bone core from the tool are unsatisfactory. The extraction of the core from the tool is performed while maintaining its grip on the rod, which is unsatisfactory and requires an additional operation of gripping the bone core in order to free it from the rod, at the risk of damaging and/or impairing its cohesion.

Other trephine guide devices have previously been proposed. For example, according to FR2739773 (MAIRE Philippe), a guide device comprises a body on which a drill bit and a tubular tool can be jointly mounted. The drill bit and the tool are essentially of the same diameter, and the drill bit is provided with a centering tip protruding from the tool to promote the positioning of the tool against the bone tissue and prevent its skidding when placed in contact therewith. The drill bit is capable of producing a pilot hole of the diameter of the tubular tool prior to said tool being placed in contact with the bone tissue, in order to promote its positioning and prevent its skidding. The tubular tool is mounted on the body by screwing, and its proximal opening is used for the transit of a piston allowing the medical practitioner to extract the removed bone core from the tool. As mentioned above, such an aiming solution is not sufficient to ensure the correct positioning of the tool when it is placed in contact with the bone tissue, and does not offer a provision for allowing the medical practitioner to conveniently manipulate the trephine in such a way to guarantee that the path it follows is the one initially planned. Moreover, the withdrawal of the bone core from the tool involves the use of a specific tool designed as a piston, which has the disadvantage of the implementation of an arrangement composed of a large number of dissociable elements and is usable independently for specific operations. The result is that such arrangements require improvements.

Also known is a prior art allowing a previously removed bone core to be implanted coaxially in a perforation made in the bone tissue and intended to receive an implant. For example, according to DE19801181 (ESSINGER HOLGER K DR), the trephine is comprised of a tubular tool and a coaxial drill bit with are driven in rotation jointly in such a way that a bone core previously removed and held in the tool can be deposited during a perforation of the bone made by means of the drill bit and intended for the reception of an implant, thus making the installation of this implant easier. More particularly, the bone core constitutes a graft that is carried by the tubular tool and deposited concomitantly with the formation of a perforation in the bone intended to receive the implant, in order to increase the graft surface in the bone tissue.

Moreover, the large diameter of the tool makes it difficult to perforate the bone tissue along the circular line of cut. The resulting efforts are such that they generate a heating of the bone tissue, making it necessary to irrigate the area where the bone tissue is to be removed, which is not always easy. Furthermore, these efforts and the delicacy of manageability of the trephine make the dimensions of the perforation made in the bone inaccurate. When the purpose of this perforation is to receive an endo-osseous implant, another specific machining tool must be used to perfect its conformation and dimensions in order to adapt it accurately to receive an endo-osseous implant.

### SUMMARY OF THE INVENTION

An object of the present invention is to propose a trephine for the removal of a bone core that is organized so as to remedy the difficulties and problems set forth above. Such a trephine is of the type comprising a tubular tool for the perforation of the bone tissue along a circular line of cutting, the axial bore of which tool is used to constitute a reserve for receiving the removed bone core. Such a trephine is also of the type comprising means of guiding the tubular tool, at least to prevent its skidding when placed in contact with the bone tissue to be perforated, and means for withdrawing the removed bone core from the tubular tool.

More particularly, the present invention seeks to propose a trephine whose manipulation is easy for the medical practitioner, especially by contributing to the positioning and guiding of the tubular tool inside the bone tissue during the perforation thereof, while still allowing an easy withdrawal and release of the removed bone core from the tubular tool, and while avoiding making the structure of the trephine complex by the optimized use of the members of which it is comprised.

The trephine of the present invention is designed for the removal of a cohesive bone core. Said trephine comprises a tool of tubular shape with circular cross section, the distal end of which is provided with at least one set of teeth to bore into the bone tissue along a circular line of cutting. This tool delimits an axial bore providing at its distal end a reserve for receiving the core upon completion of the removal, the proximal end of the trephine being provided with a gripping shaft for driving the tool in rotation by an instrument. Said trephine includes means of guiding the tool in the bone tissue comprising a stem the distal end of which protrudes from the distal opening of the axial bore of the tool and which is provided with means for centering the trephine. The protruding part of the stem constitutes a member to maintain the trephine on the bone tissue.

According to the present invention, such a trephine is primarily recognizable in that the guiding means comprise a drill bit capable of making a pilot hole in the bone tissue before the tool makes contact therewith. Said pilot hole forms a channel for receiving the protruding end of the stem that constitutes a guide finger designed as a pin capable of being inserted into such a pilot hole.

These arrangements are such that the guiding of the tool by means of the guide finger housed inside the tool is performed by means of a cylindrical surface, for the convenience of the medical practitioner in manipulating the trephine. More particularly, the drilling of the pilot hole is performed easily by means of the drill bit, accurately and without risk of skidding. The point of impact and the path of the drill bit in the bone tissue, and thus of the tool, are achieved by the drilling of the pilot hole which constitutes a guide passageway for the finger included at the end of the stem. The ease of this operation allows the medical practitioner to accurately orient the drill bit along the axis planned for removal of the bone tissue. When the pilot hole has been made, the tool is strictly and accurately guided by the guide finger.

More particularly, after the medical practitioner determines the point of impact and path of the tool, he conveniently makes a corresponding hole of small diameter by means of a drill bit. This operation to produce a pilot hole for guiding the trephine can be done easily because of the small diameter of the drill bit, indeed by starting it first with a round bur or similar instrument. When the pilot hole has been made, it forms an axially extended cylindrical channel that is used for the insertion of the guide finger protruding coaxially from the axial bore of the tool. The guide finger is particularly of cylindrical conformation with circular cross section or similar, such as polygonal cross section whose sides jointly define a circle. The operation of drilling the bone tissue by the tool is made more reliable by preventing any slippage and/or skidding of the tool on the cortical bone, and by guiding the tool along the path desired by the medical practitioner, said path corresponding to the direction of the pilot hole that was first made in the bone tissue. The presence of the guide finger, whose diameter is significantly smaller than the diameter of the tool, does not cause any damage to the core and does not diminish its cohesive nature in a single block.

The tool can be provided with the gripping shaft, and the stem can be mounted slidably inside the tool in the manner of an actuating cylinder. The piston of such an actuating cylinder can be composed of the stem equipped with a centering flange mounted slidably inside the axial bore of the tool, the cylindrical wall of which constitutes the body of the actuating cylinder.

More particularly, the trephine comprises means of coaxially centering the stem in the tool. These centering members include cooperating members made on the stem and indifferently on the tool and/or on the gripping shaft, respectively. The centering means include in particular a rod of axial extension of the guide finger, which cooperates with a guide channel made in the gripping shaft and/or at the proximal end of the tool.

The gripping shaft is preferably arranged on a guide device. It will be understood that the gripping shaft is arranged either on the tool, or on the guide device, said position of the gripping shaft varying in accordance with whether or not the trephine is organized for advantageous use with a guide device. The organization of the trephine in order to be held by the instrument for driving the tool in rotation is easily adaptable depending on the methods of organization of the guide device.

According to one advantageous embodiment, the gripping shaft is arranged on a guide device comprising detachable means of mounting the tool and/or drill bit, which means are capable of allowing a withdrawal and isolation of the tool accommodating a removed core beyond the guide device and the stem, said stem in particular being installed on the guide device.

More particularly, the relative arrangement of the mounting means and centering means imparts to the trephine the ability to allow the isolation of the tool accommodating a removed bone core, in order to facilitate the recovery of said bone core while avoiding damaging it. Because the tool is isolated, its proximal opening is completely free, forming an axial aperture offering the medical practitioner easy access for withdrawal of the removed bone core. Such a possibility of withdrawal of the tool is particularly made possible as a result of the centering means that exclude a radial obstacle made on the stem, such as a centering flange or similar interposed between the guide finger and the rod. In a case when the stem is formed from the drill bit, as set forth hereinafter, the detachable nature of the tool allows the medical practitioner to make the pilot hole prior to the installation of the tool on the guide device. These arrangements achieve an environment that is free around the drill bit during the formation of the pilot hole, thus enhancing the visual access offered to the medical practitioner.

The detachable means of mounting the tool on the guide device are, for example, of the screw-on, and/or of the interlocking or snap-on type, and/or by any other similar mounting technique allowing an easily reversible joining of the tool and the guide device. For example, the tool comprises at its proximal end a threaded zone cooperating with a complementary threaded zone made at the distal end of a body of the guide device that carries the rod. The threaded zones are respectively made to correspond on the tool and on the guide device indifferently in an internal and/or external threaded zone. Again for example, the interlocking mounting between the tool and the guide device is indifferently accomplished directly by means of cooperating interlocking members that they include, respectively, and/or indirectly by means of specific and independent cooperating mounting members.

The axial bore of the tool opens out at its proximal end to form an axial aperture offering easy access to the medical practitioner for removing the core. The advantage of the detachable mounting of the tool on the guide device is not only to allow an isolation of the tool after the core removal operation in order to facilitate the extraction thereof from the tool accommodating it, but also to make it possible to free the proximal axial opening of the tool in order to form the axial aperture. This axial aperture makes a passage for a pushing instrument, such as a cannula, a probe or similar instrument to allow the medical practitioner to easily remove the extracted core by axially pushing it out of the tool without the risk of damaging said core.

The trephine advantageously includes an axial channel for the intake of an irrigation fluid and the routing of said fluid into the axial bore of the tool. More particularly, the presence of the guide device is used to equip the trephine in a structurally simple manner for the purpose of providing it with the intake channel. This axial intake channel allows an effective irrigation of the bone tissue while it is being perforated by means of the tool. More particularly, the irrigation is done from the interior of the volume formed by the axial bore of the tool, in order to route the liquid to the heart of the zone of the bone tissue being perforated by the trephine.

According to this aspect of the present invention, the trephine advantageously integrates an axial intake channel for an irrigation fluid and for the routing of said fluid to the interior of the axial bore of the tool.

According to an advantageous arrangement of the intake channel, the centering means comprise a guide channel of the rod that is made at least in part in the interior of the gripping shaft and which is composed of the intake channel. According to a simple form of embodiment, the diameter of the guide channel is greater than the diameter of the rod in order to arrange the intake channel around said rod. According to another embodiment, the diameters of the guide channel and of the rod match, and one or more longitudinal grooves opening laterally on the guide channel are made at the periphery of said guide channel to form the intake channel. For example, the cross section of the guide channel is circular and the cross section of the rod is cruciform in conformation at least three centering branches, or of similar conformation allowing the passage of the irrigation fluid therethrough. According to another embodiment, the rod is equipped with a centering body composed of centering means and slidably mounted in the guide channel. This centering body advantageously includes orifices that comprise the intake channel for the passage of the irrigation fluid therethrough to the axial bore of the tool.

More particularly, the intake channel is formed by a guide channel for a rod that comprises the stem in extension of the guide finger. This guide channel is made in the gripping shaft and in association with the rod, comprises means of centering the stem.

In order to offer means of irrigating the bone tissue by means of the axial bore of the tool, the guide device preferably comprises a sealing member of the proximal opening of the axial bore of the tool, which makes it possible to prevent the escape of the irrigation fluid through the aperture. In order to prevent a consequent escape of the liquid from the axial bore through the lateral wall of the tool, and to ensure its routing to the zone of the bone tissue being perforated, the lateral wall of the tool preferably excludes any wide open aperture usually used by the medical practitioner for withdrawing the bone core from the tool. Fluid passages can be made through the cylindrical wall of the tool to allow the passage of liquids therethrough, particularly physiological liquids, in order to avoid an accumulation of blood and pressurization inside the axial bore.

More particularly, the guide device comprises a sealing member of the proximal opening of the axial bore of the tool.

In order to improve the convenience of use by the medical practitioner, the trephine is provided with stop means composed of a depth gauge to limit the penetration of the tool in the bone tissue. These stop means for example are composed of a stop member made on the tool, placed at a predefined distance from its distal end. The stop means are indifferently made in the axial bore of the tool and/or at the outer periphery thereof, or on the guide device. Such a stop member, for example, is composed of a flange or similar member arranged at a predetermined and/or adjustable axial distance with respect to the distal end of the tool. More particularly, and according to differing variations of embodiment, the medical practitioner either has available a set of tools each of which is provided with a stop member arranged at a respective distance from the distal end of the tool, or the stop member is provided with means of adjusting its axial position with respect to the distal end of the tool. According to a more elaborate embodiment, the trephine is provided with means of adjusting the distance of separation between the stop means and the distal end of the tool. The stop means constitute an impediment to the penetration of the tool into the bone tissue beyond a predetermined depth of drilling, in order to offer convenience of use of the tool by the medical practitioner who is spared from having to monitor the depth of penetration of the tool in the bone tissue.

According to this aspect of the present invention, the trephine advantageously comprises a depth gauge limiting the path of penetration of the tool inside the bone tissue.

According to different variations of embodiment, the stem is axially mounted indifferently slidably movable or fixed with respect to the tool. The stem can be connected to the tool by the guide device, on which the tool is detachably mounted in order to be separable from the guide device and from the stem in spite of the presence of an extracted bone core in its axial bore.

According to a first embodiment, the stem is mounted in a fixed manner on the guide device, and consequently is mounted fixed with respect to the tool, in such a way that the path of penetration of the tool inside the bone tissue is easily assessed by the medical practitioner after the guide finger reaches the bottom of the pilot hole. More particularly, the distal end of the guide finger constitutes a depth gauge limiting the penetration path of the tool inside the bone tissue.

According to a second embodiment, the stem is slidably mounted on the tool and is retractable inside the axial bore thereof under the effect of a thrust exerted at the distal end of the guide finger. More particularly, the stem is slidably mounted on the tool in such a way that it can be retracted to the interior of the bore as the tool progresses to the interior of the bone tissue. Inversely, after the removal of the core the medical practitioner can easily extract it from the tool by sliding the stem towards the exterior of the axial bore of the tool. Because the core is naturally affixed to the guide finger, said guide finger bearing the core is slidably maneuvered to the exterior of the axial bore of the tool, thus extracting it from the tool and making it easily accessible to the medical practitioner, who can simply remove it from the guide finger with no risk of damaging it.

More particularly, and according to a first variation of embodiment, the stem is mounted axially movable with respect to the tool and the depth gauge is composed of stop means indifferently made on the guide device and on the tool.

More particularly, and according to a second variation of embodiment, the stem is mounted axially fixed with respect to the tool and the depth gauge is constituted by the guide finger.

The stem is capable of being detachably mounted on the guide device, in order to allow its withdrawal. According to these arrangements, the hole made in advance by the medical practitioner is of sufficient depth to guide the tool in an operation to make a preliminary perforation in the bone tissue by means of said tool. When said operation of preliminary perforation of the bone tissue by the tool is completed, the stem is withdrawn from the guide device and the preliminary perforation is used to guide the tool into the bone tissue until the core is extracted. According to other arrangements of organization of the trephine of the invention, the presence of the pilot hole of a depth essentially corresponding to that of the perforation by the tool for the coring, results in the core, which is obtained and removed from the trephine, having an axial hole that corresponds to the pilot hole made in the bone tissue for the passage of the guide finger. According to the provisions to make a preliminary perforation, the core obtained is at least in part preserved from such an axial hole.

More particularly and according to this aspect of the present invention, the stem is detachably mounted on the guide device in such a way that the stem is able to be withdrawn after the preliminary perforation is formed by the tool.

According to a particular form of embodiment, the guide finger can be composed of the distal end of the drill bit that forms the pilot hole. Said drill bit comprises part of the guide device, so that, having been used to make the pilot hole prior to the installation of the tool on the guide device by means of the respective detachable mounting means, the drill bit is then used to guide the tool as it penetrates into the bone tissue.

The stem can be formed from the drill bit the distal end of which constitutes the guide finger. In this case, the tool is advantageously dissociable from the guide device and from the drill bit by means of its detachable mounting means on the guide device, in order to allow mounting of the drill bit alone on the guide device to make the pilot hole. The drill bit can also be mounted on the guide device by means of reversible mounting means, in such a way that it can be withdrawn to allow a penetration of the tool in the bone tissue while being guided by an impression previously made by the tool, in accordance with the aforementioned means related to the detachable nature of the stem.

In order to use the trephine of the present invention, the medical practitioner follows these steps:
making a pilot hole in the bone tissue by means of the drill bit, said hole having a diameter corresponding to the guide finger;
inserting the guide stem into the pilot hole and making the perforation of the bone tissue by means of the tool;
withdrawing the trephine and exerting a coaxial thrust on the tool against the core to remove it from the tool.

Because the trephine comprises the guide device on which the tool is detachably mounted, the medical practitioner performs an additional operation after the withdrawal of the trephine from the bone tissue, the bone core being accommodated inside the tool. This additional operation consists of withdrawing the tool accommodating the bone core by means of its detachable mounting means on the guide device, in order to dissociate the latter and the stem prior to the operation of exerting a thrust against said bone core.

The stem being mounted fixed with respect to the tool, the operation of making the perforation consists more particularly in causing a penetration of the tool in the bone tissue until the guide finger stops at the bottom of the pilot hole.

The stem being formed from a drill bit mounted detachably on the tool, the operation consisting of making the perforation comprises the following steps:
making a preliminary perforation by means of the tool until the guide finger stops at the bottom of a pilot hole of limited depth, then
withdrawing the trephine out of the bone tissue and dismounting the stem from the guide device,
making the final perforation using the preliminary perforation to center the tool in the bone tissue.

### DESCRIPTION OF THE FIGURES

The present invention will be better understood from the description of examples of embodiment, in relation to the figures on the appended plates in which:

FIGS. 1 and 2 are diagrammatic representations of a trephine of the present invention according to respective variations of embodiment.

FIG. 3 is composed of diagrams successively illustrating a method of implementing a trephine of the present invention according to the variation illustrated in FIG. 2.

FIG. 4 is composed of diagrams successively illustrating another method of implementing a trephine of the present invention according to the variation illustrated in FIG. 2.

FIG. 5 is composed of diagrams successively illustrating a method of implementing a trephine of the present invention according to the variation illustrated in FIG. 1.

FIG. 6 is composed of diagrams successively illustrating another method of implementing a trephine of the present invention according to the variation illustrated in FIG. 2.

FIGS. 7 to 9 are diagrammatical illustrations of a tool forming a trephine to make a cavity in bone tissue, according to the respective embodiments, said tool being provided with stop means limiting its path inside the bone tissue.

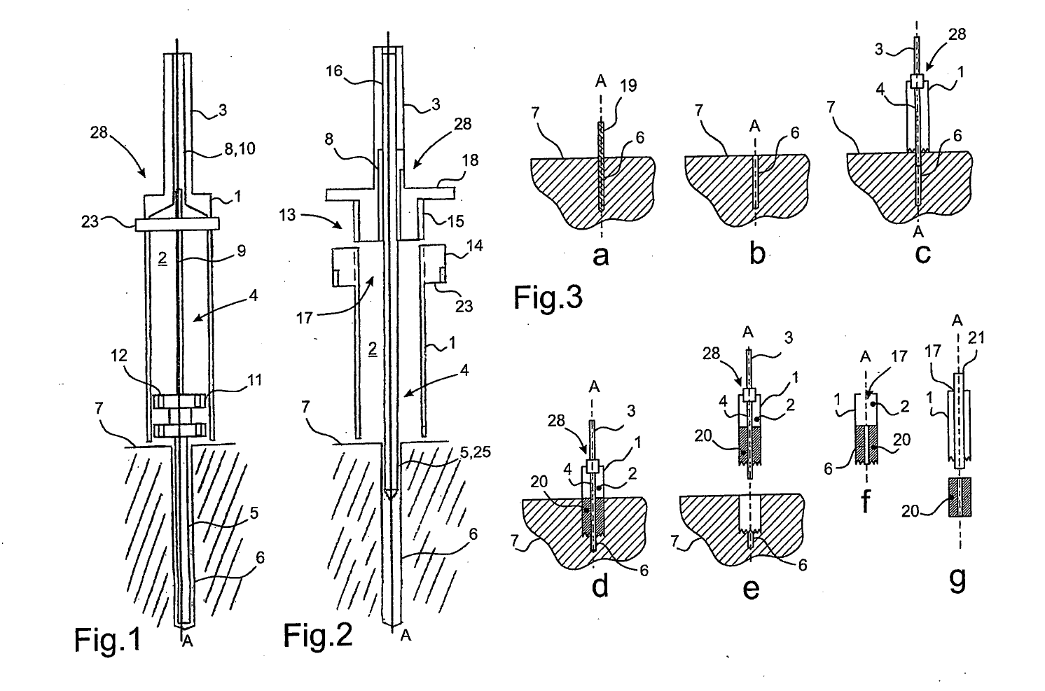

In FIGS. 1 and 2, a trephine is intended for the removal of a bone core. Such a trephine can be used in the field of bone surgery in general, and more particularly in the dental field for the installation of an endo-osseous implant. This trephine comprises a tool 1, overall cylindrical in shape, comprised of a revolution wall defining the limits of an axial bore 2 intended to accommodate the extracted bone core. Said axial bore is open at its distal end to make a passage for the core to be extracted, and the cylindrical wall of the tool is provided at its distal end with teeth for perforating the bone tissue 7 along a circular line of cut. These teeth are more particularly formed from the milling or similar method of the distal section of the cylindrical wall of the tool 1, which makes a circular line of cut defining the limits of the periphery of the core extracted and received inside the axial bore 2 of the tool 1. The advantage of an extraction of such a bone core is to allow a medical practitioner to have available a cohesive bone mass in a single block, which can be used for a graft and/or as filling. Such a trephine can also be used to produce a recess free of bone mass after perforation, and having appropriate dimensional characteristics for the reception of an endo-osseous implant, such as a dental or similar implant. The precision with which the trephine of the invention is guided makes it possible to obtain such dimensional characteristics without having to retouch the recess formed by the tool 1 in the bone tissue 7.

A gripping shaft of the trephine is provided at its proximal end for the connection by an instrument to drive the tool 1 in rotation. The tool 1 and/or the members comprising the trephine are in particular made of a biocompatible material, such as a metal or metal alloy, or a ceramic material, or a composite material combining metal and ceramic. The tool 1 and/or the other members comprising the trephine are also capable of being given a coating and/or a surface treatment. In the illustrated example of embodiment, the tool 1 has only one cutting blade for the formation of a single line of cut. According to an embodiment not represented, the tool 1 can include a plurality of axially successive cutting blades, which are made on circular cutting edges of different and progressive diameters.

The trephine is equipped with a device 28 to guide the tool 1 during the perforation of the bone tissue, comprising a guide stem 4 that protrudes coaxially out of the distal opening of the axial bore 2 of the tool 1. More particularly, the guide stem 4, the tool 1 and its axial bore 2 are coaxial with respect to a common axis A of the trephine. This guide stem 4 comprises at its distal end a guide finger 5, which is intended to be inserted inside a pilot hole 6 previously made by the medical practitioner in the bone tissue 7. The end of the guide finger 5 can be of any conformation, such as flat, rounded or conical. The insertion of the guide finger 5 inside the pilot hole 6 makes it possible to guide the contact between the bone tissue 7 and the tool 1 in order to prevent its slipping and/or skidding on the cortical bone, and allows the path of the tool 1 to be guided inside the bone tissue 7. The guide finger 5 is axially extended to form a cylinder capable of being received by a guide passage formed by the pilot hole 6.

The trephine is provided with an intake channel 8 for fluid to irrigate the zone perforated by the tool 1. Said intake channel 8 opens into the axial bore 2 of the tool 1 to spray the area of the bone tissue 7 perforated by the tool 1 and to reduce mechanical as well as heat trauma produced by said perforation and the extraction of the bone core. Alternatively, the wall of the tool 1 comprises openings for the passage of liquids therethrough, particularly physiological liquids, in order to prevent an accumulation of blood and pressurization inside the axial bore 2. The medical practitioner can also benefit from the visual access such openings provide to the area in which the perforation is being performed. The trephine is also provided with means of pushing the extracted core coaxially to the axis A of the tool 1, to allow its easy removal from the axial bore 2 while limiting the risks of damage to the core during its removal from the tool 1.

In the variation of embodiment represented in FIG. 1, the tool 1 includes the gripping shaft 3 and the guide stem 4 is slidably mounted on the trephine, and more particularly is slidably mounted inside the tool 1. Centering means allow the stem 4 to be guided while sliding in the tool 1. Said centering means utilize a guide rod 9 as a component of the stem 4 and is an extension of the guide finger 5. Said rod 9 is slidably mounted inside a guide channel 10 which is made coaxially with the axis A in the gripping shaft 3. Said guide channel 10, however, has a larger diameter than that of the rod 9, in order to constitute the intake channel 8 for the irrigation fluid. In order to prevent slack of the stem 4 inside the tool 1, the centering means also comprise a centering body 11 inside the axial bore 2 of the tool 1. If need be, said centering body 11 includes ducts 12 for the circulation of the irrigation fluid therethrough to the distal opening of the axial bore 2. The purpose of the slidable mounting of the guide stem 4 on the tool 1 is to allow its retraction to the interior of the axial bore 2 of the tool 1, under the effect of the support received by the guide finger 5 by its end at the bottom of the pilot hole 6 as the tool progresses into the bone tissue 7. Because the guide stem 4 is slidably mounted inside the tool 1, said tool is advantageously used to remove the extracted bone core by pushing it out of the tool 1.

The tool 1 is alternatively provided with a stop member 23 to limit its penetration into the bone tissue 7. This stop member 23 is particularly useful because of the axial mobility of the stem 4 intended for the retraction of the guide finger 5 into the axial bore 2 of the tool 1. More particularly, the stop member 23 constitutes a depth gauge that offers the medical practitioner means of limiting the path of penetration of the tool 1 in the bone tissue 7. Such stop member 23 is for example composed of a flange or similar member made at the outer periphery of the tool 1. The stop member 23 is made at a distance from the distal end of the tool 1 corresponding to the path of tool 1 desired by the medical practitioner in the bone tissue 7. According to a perfected embodiment not shown, the stop member 23 is provided with means of adjusting its position on the tool 1. According to other arrangements, the medical practitioner can have available a set of tools 1 comprising stop members 23 placed at respective distances with regard to the distal end of the corresponding tool 1.

In the variation of embodiment represented in FIG. 2, the trephine is shown in an exploded drawing. The guide device 28 includes the gripping shaft 3. The tool 1 is detachably mounted on the guide device 28 by mounting means 13. In the embodiment illustrated, said mounting means 13 are of the screw type and comprise a barrel 14 made at the proximal end of the tool 1 for the reception of a collared cylinder 15. The collared cylinder 15 includes the gripping shaft 3 and is provided with detachable means 16 of connection to the guide stem 4. In the embodiment illustrated, said connection means 16 are of the type by screwing the proximal end of the guide stem 4 into a housing provided for that purpose which includes the collared cylinder 15. This screw connection between the guide stem 4 and the collared cylinder 15 is advantageous. Indeed, the connection means 16 further constitute means of adjusting the length of protrusion of the guide finger 5 out of the tool 1 during the extraction operation. If need be, the guide stem 4 is interchangeable with a set of stems of different dimensions, and/or can be removed during the operation of extracting the bone core.

The proximal opening of the axial bore 2 of the tool 1 is largely open in order to form an axial aperture 17 providing an access passage to the axial bore 2 of the tool 1, for easy removal of the extracted core from the tool 1 by the medical practitioner by means of an instrument for pushing, such as a cannula or similar.

When the tool 1 is mounted on the guide device 28, the collar of the cylinder 15 forms a sealing member 18 of the proximal opening 17 of the axial bore 2 of the tool 1. The irrigation channel 8 is made in the guide device 28, and more particularly in the collared cylinder 15 and at least in part in the gripping shaft 3. The intake channel 8 opens coaxially into the axial bore 2 of the tool 1. In the embodiment illustrated, such an intake channel 8 can comprise a lateral orifice to connect with an irrigation fluid feed provided with a swivel fitting to connect to a fluid feed source.

According to a particular form of embodiment, the guide finger 5 is designed as a drill bit to perforate the bone tissue 7. More particularly, the pilot hole 6 is made by the medical practitioner by means of a drill bit 25 as part of the guide device. The pilot hole 6 is made prior to the installation of the tool 1 on the guide device. Said pilot hole 6 is in particular of shallow depth, that is, a sufficient depth to guide the drill bit 25 in a subsequent step after the installation of the tool 1 on the guide device by mounting means 13. More particularly, the pilot hole 6 is made at a shallow depth by means of the drill bit 25 installed on the guide device. The tool 1 can already be installed on the guide device, but it only comes into contact with the bone tissue 7 after the formation of the pilot hole 6. According to other ways of utilization of the trephine, the drill bit 25 [is] mounted reversibly or integrally with the guide device 28, and is used to form the pilot hole without the presence of the tool 1. The drill bit is then withdrawn from the pilot hole 6 and the tool 1 is installed on the guide device 28. The tool 1 is then moved near to the bone tissue 7 and the drill bit 25, constituting the guide finger 5, is inserted into the pilot hole 6. The medical practitioner then performs the perforation of the bone tissue 7 by means of the tool 1. After formation of the pilot hole 6, the drill bit 25 perforates the bone tissue 7 as the tool penetrates therein. These arrangements make it possible to reduce the tools needed by the medical practitioner, by using the drill bit 25 for the formation of the pilot hole as well as the guide finger 5.

The provisions for using the drill bit 25 as guide finger 5 prevent the use of the drill bit 25 as depth gauge. In order to increase the convenience of use of the trephine by the medical practitioner, the tool 1 is equipped with means 23 to stop its penetration into the bone tissue 7 beyond a predefined depth. The stop means 23 are advantageously composed of the distal collar of the barrel 14 comprising mounting means 13. The axial extension of the barrel 14 can be adapted to place the collar comprising the stop means 23 at a predefined distance from the distal end of the tool 1, corresponding to a depth of penetration of the tool 1 into the bone tissue 7. Such an adaptation can be made either permanently in order to place the stop means 23 at a suitable sampling distance from the distal end of the tool 1, or by means of adjusting the axial position of the stop means 23.

In FIG. 3, a method of implementation of a trephine of the present invention consists, in general terms, in carrying out the following steps:
producing in the bone tissue 7 a pilot hole 6 of a diameter corresponding to the guide stem 4, and more particularly to that of the guide finger 5, as illustrated in diagram (a). This operation can consist more particularly of preparing a starter hole in the cortical bone by means of a round bur or similar instrument, in such a way that the actual point of impact of the perforation for coring the bone tissue 7 is most accurately and easily accomplished. A drill bit 19 of small diameter is then used to form the pilot hole 6. The smallness of the diameter of the pilot hole 6 is determined as a function of the diameter of the guide finger 5, and with regard to the diameter of the tool 1 which is significantly larger than the diameter of the guide finger 5. In diagram (b) the bone tissue 7 includes a pilot hole 6 ready to receive the guide finger 5 of the trephine.
inserting the trephine's guide stem 4 into the pilot hole 6, as illustrated in diagram (c). Because the tool 1 is guided by means of the guide stem 4, any risk of slippage and/or skidding of the tool 1 on the cortical bone is avoided. Since the guide stem 4 is coaxial with the tool 1, the path of the tool 1 will correspond exactly with that of the drill bit 19 used to form the pilot hole 6. The bone tissue 7 is then perforated by means of the tool 1, as shown in diagram (d).
withdrawing the trephine as shown in diagram (e). The bone core 20 is held in the axial bore 2 of the tool 1. A thrust coaxial to the tool 1 is then applied against the bone core 20 to remove it from the tool 1, as shown in diagram (g).

FIG. 3 illustrates in a general way a method of implementation of a trephine of the present invention. However, specific provisions for the use of a trephine of the type shown in FIG. 2 are also illustrated in this figure. The operation consisting of performing the perforation consists more particularly in causing the tool 1 to penetrate into the bone tissue 7 until the guide finger 5 stops at the bottom of the pilot hole 6. The guide finger 5 then constitutes a depth gauge of the penetration of the tool 1 into the bone tissue 7, as represented in diagrams (c) and (d). In particular, and irrespective of the previous provisions, the operation consisting of exerting a thrust against the core 20 comprises more particularly the steps consisting of:
dismounting the tool 1 from the guide device 28 in order to clear the aperture 17 as illustrated in diagram (f), and
exerting an axial thrust against the core 20 by means of a cannula 21 or similar instrument, which is inserted into the axial bore 2 of the tool 1 through the aperture 17, as illustrated in diagram (g).

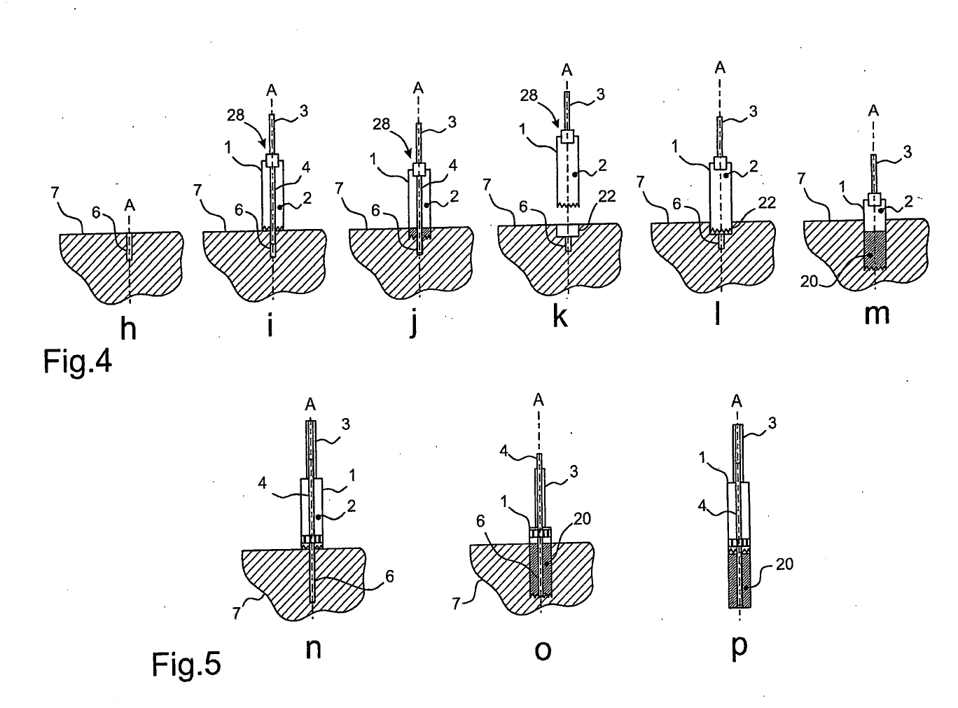

FIG. 4 illustrates a specific possible implementation of a trephine of the type shown in FIG. 2. In particular, the operation consisting of performing the perforation comprises the steps consisting of:
making a pilot hole 6 of shallow depth in the bone tissue 7 in the manner described in FIG. 3, as illustrated in the diagram (h). Said pilot hole 6 is intended to be used for an intermediate step consisting of making a preliminary perforation 22 by means of the tool 1 until the guide finger 5 stops at the bottom of a pilot hole 6 of limited depth, as illustrated in diagrams (i) and (j)
then withdrawing the trephine, and in particular the tool 1, out of the bone tissue 7 and dismounting the stem 4 from the guide device, as illustrated in diagram (k).
then performing the final perforation by using the preliminary perforation 22 to center the tool in the bone tissue 7, as illustrated in diagrams (l) and (m).

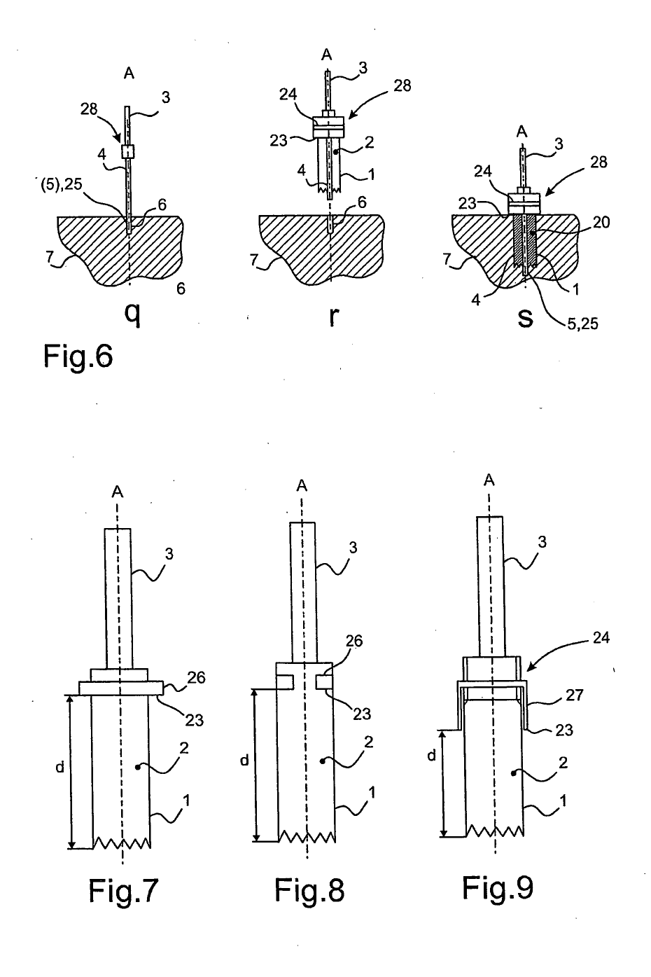

FIG. 6 illustrates another specific possible implementation of a trephine of the type represented in FIG. 2. A drill bit 25 is installed on and/or is made integral with the guide device 28, said guide device not carrying the tool 1. A pilot hole 6 of shallow depth is made in the bone tissue 7 by means of the drill bit 25, as illustrated in diagram (q). The drill bit 25 is then withdrawn from the pilot hole 6 to allow the mounting of the tool 1 on the guide device 28, as illustrated in diagram (r). The distal end of the drill bit 25 is then inserted into the pilot hole 5 in order to constitute the guide finger 5, and the bone tissue 7 is perforated by the tool 1, as illustrated in diagram (s). During this operation, the drill bit 25 progresses into the bone tissue 7, perforating it concomitantly and in advance of the progression of the tool 1 into the bone tissue 7.

With reference again to FIG. 6, the trephine is provided with stop means 23. More particularly, the tool 1 and/or the guide device 28 comprise a stop member composed of said stop means 23 and formed by a flange or similar member. The path of penetration of the tool 1 into the bone tissue 7 is limited by the stop means 23, as illustrated in diagram (s), in such a way that the medical practitioner is spared from the need to monitor the extent of the penetration of the tool 1 into the bone tissue 7. The stop means 23 are arranged at a predefined distance from the distal end of the tool 1. According to several variations of embodiment, either the trephine is chosen by the medical practitioner from among a set of trephines having stop means 23 placed at [a] respective distance from the distal end of the tool 1, or the trephine is equipped with means 24 of adjusting said distance, as in the embodiment represented.

FIG. 5 illustrates details of implementation of a trephine of the type shown in FIG. 1. More particularly, the operation of making the perforation consists of producing a penetration of the tool 1 in the bone tissue 7 until the guide finger 5 is completely retracted into the axial bore 2 of the tool 1, as illustrated in diagrams (n) and (o). Again in particular, the operation of exerting a thrust against the core 20 consists of producing a thrust against the stem 4 in order to evacuate the guide finger 5 out of the axial bore 2 of the tool 1, to which guide finger 5 the core 20 is naturally attached. After the core 20 has been extracted from the tool 1, it is easily accessible for the medical practitioner.

In FIGS. 7 to 9, a trephine incorporates stop means 23 in order to limit the axial penetration of the tool 1 into the bone tissue 7 to a predetermined path. This limitation of path is achieved by a placing the trephine in axial stop against the cortical bone. In the illustrated forms of embodiment, the trephine is composed of the trephine 1 and the gripping shaft 3. Such a trephine may not incorporate a guide device 28 of the tool 1 during penetration of the bone tissue, as previously described.

In the examples of embodiment represented in FIGS. 7 and 8, the stop means 23 are composed of the distal support of a flange 26 or similar included in the tool 1. Said flange 26 is made either at the outer periphery of the tool 1, as illustrated in FIG. 7, or inside the axial bore of the tool 1, as represented in FIG. 8. The stop means 23 are placed at a predefined distance d from the distal end of the tool 1.

In the example of embodiment represented in FIG. 9, the stop means 23 are composed of the distal end of a stop member 27 mounted axially movable on the tool 1. Said stop member 27 is for example composed of a sleeve screwed onto the tool, the distal end of which constitutes the stop means 23. The movable mounting of the stop member 27 makes it possible to adjust the distance d of separation between the stop means 23 and the distal end of the tool 1. The means of moving the stop member 27 constitute means 24 of adjusting said distance d. These adjustment means associate for example a slidable mounting of the stop member 27 on the tool, and means of immobilizing the stop member 27 in a desired axial position. In the example of embodiment illustrated, the adjustment means 24 are constituted by a mounting of the stop member 23 onto the tool 1 by screwing; said adjustment means can be completed by immobilization means not shown. Such immobilization means are, for example, composed of a specific immobilization member attached both to the tool 1 and to the stop member 27, or by a stiffening of the mobility of the stop member 27 by means of a flexible mass interposed between the stop member 27 and the tool 1.

1. Trephine for removal of a bone core, said trephine comprising a tool of tubular shape with circular cross section, the distal end of which is provided with at least one set of teeth to bore into the bone tissue along a circular line of cutting, said tool delimiting an axial bore providing at its distal end a reserve for receiving the core upon completion of the removal, the proximal end of the trephine being provided with a gripping shaft for driving the tool in rotation by an instrument, said trephine including means of guiding the tool in the bone tissue comprising a stem the distal end of which protrudes from the distal opening of the axial bore of the tool and which is provided with centering means of the trephine, the protruding part of the stem constituting a member to maintain the trephine on the bone tissue, wherein the guiding means comprise a drill bit capable of making a pilot hole in the bone tissue before the tool makes contact therewith, said pilot hole forming a channel for receiving the protruding end of the stem that constitutes a guide finger designed as a pin capable of being inserted into such a pilot hole.

2. The trephine of claim 1, wherein the gripping shaft is arranged on a guide device comprising detachable means of mounting the tool, which means are capable of allowing a withdrawal and isolation of the tool accommodating a removed core beyond the guide device and the stem.

3. The trephine of claim 1, wherein it includes an axial channel for the intake of an irrigation fluid and the routing of said fluid into the axial bore of the tool.

4. The trephine of claim 3, wherein the intake channel is formed by a guide channel for a rod that comprises the stem in extension of the guide finger, the guide channel being made in the gripping shaft and in association with the rod, comprising means of centering the stem.

5. The trephine of claim 2, wherein the guide device comprises a sealing member of the proximal opening of the axial bore of the tool.

6. The trephine of claim 1, wherein it Comprises a depth gauge limiting the path of penetration of the tool into the bone tissue.

7. The trephine of claim 1, wherein the stem is indifferently mounted slidably movable or fixed with respect to the tool.

8. The trephine of claim 6 wherein the stem being axially movable with respect to the tool, the depth gauge is constituted by stop means indifferently made on the guide device and on the tool.

9. The trephine of claim 6 wherein the stem being axially fixed with respect to the tool, the depth gauge is constituted by the guide finger.

10. The trephine of claim 2 wherein the stem is detachably mounted on the guide device, in such a way that the stem is capable of being withdrawn after the formation of a preliminary perforation by means of the tool.

11. The trephine of claim 2 wherein the stem is formed from the drill bit the distal end of which constitutes the guide finger.

12. A method of using a trephine of claim 1, comprising performing the following steps:
making a pilot hole in the bone tissue by means of the drill bit of a diameter corresponding to the guide finger;
inserting the guide stem into the pilot hole and making the perforation of the bone tissue by means of the tool;
withdrawing the trephine and exerting a coaxial thrust on the tool (1) against the core to remove it from the tool.

13. The method of claim 12, comprises comprising an additional operation consisting of withdrawing the tool accommodating the bone core in order to dissociate the guide device and the stem, prior to the operation consisting of exerting a thrust against said bone core.

14. The method of using a trephine as claimed in claim 12 wherein the operation consisting of making the perforation consists in causing a penetration of the tool into the bone tissue until the guide finger stops at the bottom of the pilot hole.

15. The method of claim 12 wherein the operation consisting of making the perforation comprises the steps consisting of:
making a preliminary perforation by means of the tool until the guide finger stops at the bottom of a pilot hole of limited depth, then
withdrawing the trephine out of the bone tissue and dismounting the stem from the guide device,
making the final perforation using the preliminary perforation to center the tool in the bone tissue.
